(19) United States
(12) Patent Application Publication
(10) Pub. No.: US 2021/0138218 A1
(43) Pub. Date: May 13, 2021

(54) ULTRASOUND-RESPONSIVE CONTAINERS FOR DRUG DELIVERY

(71) Applicant: BIONAUT LABS LTD., Herzliya (IL)

(72) Inventors: Michael SHPIGELMACHER, Los Angeles, CA (US); Alex KISELYOV, San Diego, CA (US); Sergey KOLOTILOV, Kiev-80 (UA); Oleksiy SHVETS, Kiev (UA); Michael KARDOSH, Kiryat Ono (IL); Eran OREN, Tel Aviv (IL)

(73) Assignee: BIONAUT LABS LTD., Herzliya (IL)

(21) Appl. No.: 17/052,201

(22) PCT Filed: Nov. 2, 2018
(86) PCT No.: PCT/US18/59020

§ 371 Date: Nov. 1, 2020

Related U.S. Application Data
(63) Continuation-in-part of application No. PCT/US2018/030953, filed on May 3, 2018.

(51) Int. Cl. A61M 37/00; A61K 38/46; A61K 31/7105; A61K 48/00; A61K 41/00; A61K 9/00

(52) U.S. Cl. CPC A61M 37/0092; A61K 38/465; A61K 31/7105; A61K 48/0075; A61K 41/0047; A61K 9/0009; A61M 2205/0288; A61M 2202/0413; A61M 2202/0405; A61M 2202/0403; A61M 2202/0464; A61M 2205/0238; A61M 2207/00; A61K 9/0024

(57) ABSTRACT

This invention relates to coated mesoporous nanoparticles (MSN). The coating material is an ultrasound-responsive material (for example a polymer) and it acts as a control layer for blocking/release of material loaded in the pores of the MSN.

### CROSS REFERENCE TO RELATED APPLICATIONS

This application claims the benefit of International Serial No. PCT/US2018/030953, filed on 3-May-2018 (and entitled ‘Triggering of Payload Release From Miniaturized Devices’) which is incorporated in their entirety herein by reference.

### FIELD OF THE INVENTION

This invention relates to coated mesoporous nanoparticles (MSN). The coating material is an ultrasound-responsive material (for example a polymer) and it acts as a control layer for blocking/release of material loaded in the pores of the MSN.

### BACKGROUND OF THE INVENTION

Ultrasound (US)-based methods currently exist for remotely triggering release of a medical payload, such as drugs and diagnostic aids, from particles or devices implanted in a living tissue. US Remotely-triggered payload release from particles or implantable devices have been researched in the past. The purpose of such methods is to generate an external trigger for payload release (drug or diagnostics) from a carrier housing such a payload (e.g., particle or implantable device) in a living tissue. Remotely-triggered payload release is desirable in supporting specific clinical goal, such as:
Release of a medical payload only when the carrier particle is in the right location for treatment (e.g., tumor);
Release of a medical payload only when the moment is right (e.g., in the middle of a clinical procedure); or
Release of a medical payload in a time-dependent or stop-and-go manner to treat predetermined area(s) for a predetermined time.

Existing US-based trigger methods rely on a variety of effects, including:
Thermal/mechanical effect based on cavitation (leading to localized heating due to vibration and increased speed of diffusion and/or changes in localized chemical characteristics which increase diffusion);
Mechanical degrading/rupturing of carrier leading to payload release;
Shape change of the carrier or an integrated part thereof; or
Change to the characteristics of the surrounding biological tissue into which the payload is being released (e.g., sonoporation), resulting in improved payload diffusion/absorption through tissue.

A common drawback of these methods is that each method supports only a subset of the typical technical features desired from a clinical standpoint. These features for an ultrasound-based remote trigger system for clinical payload release include:
Customizable tissue penetration depth (10 cm or greater) to be able to trigger payload release in deeply situated tissue. For instance, release methods in the >7 MHz (diagnostic US) range are typically limited to less than 10 cm penetration.
Customizable frequency range (KHz-MHz range) to offer compatibility with existing medical ultrasound equipment and to minimize invasiveness to tissue. For example, cavitation-based methods are typically most effective in the KHz range using high intensity focused ultrasound (HIFU), while polymer degradation-based methods are more effective in the MHz range (diagnostic US);
Support for gradual payload release over a controllable time period, or an on-off switchable release functionality (rather than a single release pulse). For example, methods relying on degradation of a uniform polymer encasing the payload are by design irreversible and do not have gradual release functionality; and
Individual control of multiple payload carriers in a single tissue volume unit (e.g., releasing payload selectively from only a single particle out of many located within the same organ). None of the existing methods offer this functionality.

It would therefore be desirable to have implantable devices and methods thereof, which overcome the above restrictions of the current capabilities. This goal is attained by embodiments of the present invention.

### SUMMARY OF THE INVENTION

In one embodiment, this invention provides a carrier device that can be implanted in a tissue, the carrier device comprises a porous structure. The pores of the porous structure comprise a functional material that can be released in a specific location in the body upon demand The functional material can be any material that is used for treatment or diagnostics. For example, the functional material can be a drug, a binder for a biological material, an imaging element etc. In order to control the release of the functional material from the porous structure, the porous structure is coated by a material that is sensitive to ultrasound (US). The material is chosen such that when release of material from the porous structure is not required, the coating material assume a dense/closed structure that blocks release of materials from the pores of the porous structure. However, when release of materials from the pores of the porous structure is required, the coating material changes its conformation to allow such release. This change of conformation is induced by applying ultrasound to the coating material. Accordingly, control of substance release from the porous structure is achieved using an external US source that can be turned on and off upon demand.

In one embodiment, this invention provides a carrier device for implanting in a biological tissue for precise delivery and release of a functional material in said tissue or in another tissue, the carrier device comprising:
a porous particle coated by US-sensitive coating material;
a functional material; and
a propelling component;
   wherein, the functional material resides in the pores of the porous material; and wherein the propelling component is attached to the porous particle.

In one embodiment, the propelling component is a magnetic component. In one embodiment, the coating material is a polymer. In one embodiment, the propelling component and the coating materials are responsive to external stimuli.

In one embodiment, the coating material is sensitive to ultrasound (US) stimuli; and the propelling component is responsive to stimuli selected from US, magnetic, electric, electromagnetic, thermal, electromagnetic radiation or a combination thereof.

In one embodiment, the application of the stimuli to the propelling component propels the device. In one embodiment, the US-sensitive material undergoes a chemical or structural modification in response to US.

In one embodiment, the chemical or structural modification comprises a polymer decomposition or change from coil conformation to globular conformation structure. In one embodiment, the US-sensitive coating material changes its chemical structure, length, molecular weight, shape or topology or detaches from the particle or ruptures or becomes perforated in response to the external US stimuli. In one embodiment, the porous material is silica or alumina.

In one embodiment, the wherein the average size of the porous particle ranges between 10 nm and 1000 nm. In one embodiment, the average size of the porous particle ranges between 100-150 nm or between 100-200 nm or between 50-100 nm or between 10-50 nm or between 2-50 nm or between 60-130 nm or between 70-150 nm or between 30-60 nm.

In one embodiment, the BET specific surface area of the porous particle ranges between SBET 817-1044 m²/g. In one embodiment, the total volume of the pores of the porous particle ranges between 0.9 cm³/g and 1.4 cm³/g. In one embodiment, the pore diameter ranges between 3 nm and 10 nm.

In one embodiment, the frequency of the US ranges between 10 and 40 KHz. In one embodiment, the frequency of the US is 20KHz. In one embodiment, the US-sensitive material does not undergo structural modification in response to US in a MHz frequency range.

In one embodiment, the functional material is an organic compound, a polymer, a composite or a combination thereof. In one embodiment, the functional material comprises small molecules, biological materials, gene therapy components, antisense oligonucleotides, aptamers, peptides, peptoids, endogenous or engineered cells, oncolytic viruses or radiation therapy materials.

In one embodiment, the gene therapy components comprise CRISPR/Cas9 or viral vector-based agents. In one embodiment, the particle is a microparticle, a nanoparticle or a combination thereof. In one embodiment, the polymer is a copolymer, comprising or consisting of: 2-(2-methoxyethoxy)ethylmethacrylate and tetrahydropyranyl methacrylate-poly(2-(2-methoxyethoxy)ethylmethacrylate-co-2-tetrahydropyranyl methacrylate), p(MEO2MA-co-THPMA).

In one embodiment, the polymer comprises a carboxy end group. In one embodiment, the porous particle comprises (3-aminopropyl) triethoxysilane (APTES) and the carboxy group of the polymer binds to the NH₂ group of the APTES for covalent immobilization of the polymer on the porous particle.

In one embodiment, the polymer is temperature sensitive. In one embodiment, the polymer changes conformation in response to a temperature change In one embodiment, the conformational change comprises coil structure at temperatures below 20-30° C. and globules structure at temperatures above 20-30° C.

In one embodiment, this invention provides a system comprising:
the device of claim 1; and
a remote unit;
   wherein the remote unit is configured to apply external stimuli to said device.

In one embodiment, the external stimuli comprise US. In one embodiment, the coating material changes its shape or topology, or detaches from the particle ruptures or becomes perforated in response to the external stimuli; or
the propelling component is driven in response to the external stimuli; or a combination thereof.

In one embodiment, this invention provides a method for operating a device, the method comprising:
providing a carrier device comprising:
   a porous particle coated by US sensitive coating material;
   a functional material; and
   a propelling component;
      wherein, the functional material resides in the pores of the porous material; and wherein the propelling component is attached to the porous particle;
applying external stimuli to the device.

In one embodiment, the coating is responsive to the external stimuli. In one embodiment, the stimuli is US, magnetic or a combination thereof.

In one embodiment, the coating polymer changes its chemical structure, molecular weight, shape or topology, or detaches from the particle ruptures or becomes perforated in response to the external stimuli, such that the functional material is released from the particle in response to the external stimuli; or the propelling component is driven in response to the external stimuli; or a combination thereof.

In one embodiment, the coating material is responsive to US and the propelling component is responsive to magnetic stimuli; or the coating material is responsive to US of a first frequency and the propelling component is responsive to US of a second frequency.

In one embodiment, the functional material is an organic compound, a polymer, a composite or a combination thereof. In one embodiment, the coating material comprising a polymer.

In one embodiment, the porous particle is a microstructure, a nanostructure or a combination thereof. In one embodiment, the propelling component comprises a magnetic component.

In one embodiment, this invention provides a method of producing a carrier device of this invention, the method comprising:
providing or constructing a porous particle;
filling the pores of the porous particle with a functional material;
coating the porous particle with a US-sensitive coating material.
binding a propelling component to the porous particle, to the coating material or to a combination thereof.

In one embodiment, the invention provides a method of treating a subject, the method comprises:
inserting the carrier device of this invention into the subject;
applying external stimuli to the device.

In one embodiment, inserting the device comprises inserting the device into a certain tissue within the subject.

In one embodiment, the external stimuli comprise:
magnetic/electric, acoustic, ultrasonic or electromagnetic stimuli to propel the device to a defined location within the subject; or
US stimuli to induce release of the functional material from the porous particle; or
a combination thereof.

In one embodiment, following application of the external stimuli for release of the functional material, the functional material interacts with the tissue or with component(s) of/in the tissue. In one embodiment, the interaction results in a therapeutic effect, a diagnostic effect or a combination thereof.

In one embodiment, the method further comprising imaging the location of the device within the subject. In one embodiment, the propelling component is a magnetic component.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts TEM images of MSNs-1 (a), MSNs-2 (b), MSNs-3 (c), MSNs-4 (d) samples.

FIG. 2 shows particle size distributions for MSNs samples (according to DLS data)

FIG. 3 shows (a) carbon hydrogen nitrogen (CHN) analysis data for MSNs samples and (b) N₂ adsorption isotherms (at 77K) for MSNs-1 (1), MSNs-2 (2), MSNs-3 (3), MSNs-4 (4) and MSNs-5 (5) samples.

FIG. 4 shows spectra of diffuse reflection in visible range (a) and fluorescence at excitation wavelength 470 nm (b) for Rhodamine B-labeled mesoporous silica nanoparticles MSNs-5

FIG. 5 is an estimation of monomers ratio in co(MEO₂MA/THPMA) by NMR signals integration.

FIG. 6 shows DLS patterns of copolymers co(MEO₂MA/THPMA): 1-0.875/0.125, 2-0.850/0.150, 3-0.885/0.115

FIG. 7 shows GPC RID analysis data for copolymer-1.

FIG. 8 shows the determination of phase transition temperature (lower critical solution temperature, LCST) for polymer 1.

FIG. 9 shows release profiles of fluorescein from hybrid-MSNs in PBS solution versus time with US exposure (20 KHz) and without US.

FIG. 10 shows release profiles of fluorescein from hybrid-MSNs or pure MSNs in PBS solution versus time with US exposure (20 KHz,) and without US.

FIG. 11 is ¹H NMR spectra of copolymer-2 before and after US treatment.

FIG. 12 shows release profiles of fluorescein from MSNs in PBS solution versus time with US exposure (20 kHz) and without US for scaled samples.

FIG. 13 shows fluorescein release profiles for MSN-6 samples in PBS solution versus time with US exposure (1MHz, 8W or 20 kHz, ˜14 W) and without US.

FIG. 14 Shows fluorescein release profiles for MSN-6 samples in PBS solution versus time with US exposure (1MHz, 8W—at different t or 20 kHz, ˜14 W) and without US.

FIG. 15 is a scheme of US focusing lens (a) and visualization of 1 MHz ultrasound focused by spherical lens with R=3 cm (b).

FIG. 16 is TEM images of MSN-1 sample (mesopore ordering with d˜4.5 nm, Dₚₒᵣₑ˜2.8 nm is shown).

FIG. 17 shows XRD patterns of MSNs samples.

FIG. 18 shows Thermal analysis of MSNs samples

FIG. 19 (a) (b) Intra-particle (19a) and inter-particle (19b) pore size distribution for MSNs-1 (1), MSNs-2 (2), MSNs-3 (3), MSNs-4 (4) and MSNs-5 (5) samples calculated from N₂ adsorption isotherms.

FIG. 20 are ¹H NMR spectra of THPMA (monomer), MEO₂MA (monomer) and co(MEO₂MA/THPMA)-1.

FIG. 21 are ¹H NMR spectra of THPMA (monomer), MEO₂MA (monomer) and co(MEO₂MA/THPMA)-2.

FIG. 22 are ¹H NMR spectra of THPMA (monomer), MEO2MA (monomer) and co(MEO2MA/THPMA)-3.

FIG. 23 are CHN analysis of THPMA and copolymers 1, 2 and 3.

FIG. 24 are FTIR spectra of monomers and copolymer-1

FIG. 25 shows ¹H NMR spectra of monomers and “shortened” copolymer-6

FIG. 26 shows GPC RID analysis data for copolymer-1 and copolymer-2.

FIG. 27 a) and b) are XRD pattern of MSN samples covered by copolymers 1, 2, 6 and 7.

FIG. 28 a) and b) FTIR spectra of MSN samples covered by copolymers 1, 2, 6 and 7.

FIG. 29 is a calibration graph for determination of fluorescein content in the solution. The dependence of fluorescein luminescence on its concentration is not linear and better described by polinom. Fluorescein: λ_{exc} 490, λ_{cm} 514 nm.

FIG. 30 shows concentrating lens for 1 MHz US.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment of this invention, silica mesoporous nanoparticles (MSN) with immobilized carboxylic acid-terminated temperature and ultrasound (US)-responsive polymer were prepared. In some embodiments, the MSNs were labeled with Rhodamine-B for visualization thereof and potential estimation of particles content by luminescence spectroscopy, while the polymer acted as a layer which controlled blocking/release of dye (fluorescein), loaded in pores. Based on our internal data, the polymer adopted coil conformation at T<ca. 20° C., while temperature growth led to change of conformation and formation of globules on the surface of MSN. Thus, the cargo was loaded in the polymer-grafted MSNs at low temperatures, and heating to room temperature led to pores closing. Treatment of the dye-loaded polymer-grafted MSNs with 20 kHz ultrasound followed by washing with phosphate buffer solution led to fluorescein release. For the best sample, 3-fold increase of fluorescein release upon US-treatment compared to control sample (no US treatment) was achieved using 20 kHz US. The mechanism of pores opening under UV irradiation was studied to reveal that the treatment with 20 kHz US caused pores opening due to the chemical breaking (hydrolysis) of the polymer.

The study was based on the idea that pores in the inert carriers, such as mesoporous silica particles, could be “plugged” by a material, which undergoes change of conformation under action of ultrasound (US). In one example, the ultrasound-responsive polymer used was copolymer of 2-(2-methoxyethoxy)ethylmethacrylate and tetrahydropyranyl methacrylate-poly(2-(2-methoxyethoxy)ethylmethacrylate-co-2-tetrahydropyranyl methacrylate), p(MEO2MA-co-THPMA). This polymer was chosen as such “plug”, because it is known to be able to change conformation under US treatment. Polymerization was initiated by 4,4′-Azobis(4-cyanovaleric) acid, bearing carboxy-group (Scheme 1). The presence of this carboxy-group is critically important to this embodiment, because it is bound to NH₂ group of (3-aminopropyl)triethoxysilane (APTES) for further covalent immobilization on MSNs.

[chemical structure]

As an example, mesoporous silica nanoparticles (MSNs) were chosen as the basis for composite creation due to simplicity of synthesis and surface modification, stability in various solutions at wide range of pH values (all range except alkaline media), large size of pores sufficient for sorption of large molecules along with high accessible volume.

Mesoporous nanoparticles, MSNs, were synthesized by controlled hydrolysis of the silica source (tetraethylorthosilicate TEOS) in the presence of template (cetyltrimethylammonium bromide, CTAB). In order to reveal the influence of synthetic conditions on the size and porosity of MSNs, four (4) distinct samples of the particles were prepared varying mean particles size and ordering (labeled as MSN-1 to MSN-4, while MSN-5 indicates Rhodamine-B labeled particles, vide infra). Application of a previously described protocol led to the formation of MSNs with pore sizes of 100-150 nm (MSN-1). 2-fold increase of TEOS concentration or 1.5-fold increase of concentration of all reagents or a respective decrease in water content resulted in formation of a smaller MSNs (60-130 nm and 70-150 nm, respectively—MSN-2 and MSN-3). 2-fold decrease in concentration of all reagents or dilution of the reaction mixture also yielded smaller MSNs featuring particles of 30-60 nm in size (MSN-4). The formation of mesopores in all samples was confirmed by high-resolution TEM (FIG. 16) and powder X-ray diffraction analysis (FIG. 17).

As shown by TEM, in all cases MSNs had almost spherical shape; the content of particles aggregates was low (FIG. 1). The size of MSNs in colloid solutions was verified by dynamic light scattering (DLS). There was good correlation between the sizes determined via DLS and TEM protocols for samples MSN-1 and MSN-2 (see FIGS. 1 and 2). At the same time, the sizes of particles in samples MSN-3 and MSN-4 measured by DLS appeared to be larger than those measured by TEM. The difference can be explained by formation of aggregates in colloid solution.

The method which led to high-ordered MSNs with ca. 100-150 nm average size (MSN-1) was selected as a basic method for the synthesis of Rhodamine-B labeled sample. Rhodamine-B was incorporated in the silica particles by covalent immobilization via thiocyanate group namely by treating the particles with (3-aminopropyl)triethoxysilane (APTES). This labeling was introduced for visualization of the MSNs and estimation of particles content by luminescence spectroscopy.

Samples of MSNs contained ca. 32-43% of CTAB template and up to 15% of water as calculated from the data of C, H, N analysis (FIG. 3a) and thermal gravimetry (FIG. 18). This template was located in pores and had to be removed for formation of samples by ionic exchange using a solution of ammonium nitrate in ethanol (95%) at 70° C. It was found that this method did not allow to eliminate all template from pores (FIG. 3), however the procedure of ionic exchange (in contrast to calcination at 500° C.) was selected in order to preserve Rhodamine-B as well as to avoid particles aggregation on heating.

Porous characteristics of MSN samples were determined by N₂ adsorption measurements at 77 K (FIG. 3b). All samples possessed high porosity, specific surface areas SBET varied in range 817-1044 m²/g, the values of total pore volume were in range 0.9-1.4 cm³/g. As expected, there was significant contribution of mesopores in total sorption capacity. For example, sample MSN-5 (Rhodamine-labeled sample) had S(mesopores)=915 m²/g and total specific surface 989 m²/g, volume of mesopores 0.90 cm³/g and total pores volume 1.09 cm³/g, and mesopores diameter 2.91±0.24 nm (calculation of pore diameters are shown in FIG. 19).

It was found that optical characteristics of Rhodamine-B in MSN-5 strongly depended on the presence of template (CTAB) in pores. Template removal led to the absorption (reflectance) maxima shift and change of luminescence intensity. We concluded that luminescence of Rhodamine cannot be used for quantitative determination of the guests content in pores, because its own luminescence would probably vary upon guests loading.

In order to accomplish MSN coating, three polymer batches were prepared using varying monomer ratios of: 2-(2-methoxyethoxy)ethylmethacrylate/2-tetrahydropyranyl methacrylate (MEO₂MA/THPMA) contents were set to 0.875/0.125 (polymer 1), 0.850/0.150 (polymer 2) or 0.885/0.115 (polymer 3). Polymer composition was studied by NMR spectroscopy. It was shown that polymerization was complete, no traces of monomers were found by NMR (FIGS. 20-22). At the same time, the polymer samples contained impurity of DMF (deemed to be acceptable since it was added as a solvent for the next stage) and n-hexane (used for purification). Carboxyl group of initiator was not detected by NMR (its expected content was ca. 0.003 μmol/0.01 mmol of monomers). Formation of the polymer was also conformed by C,H,N analysis (FIG. 23) and IR-spectroscopy. All specific bands characteristic of the starting monomers except for the C═C double bonds (as expected) were found in IR-spectra of the polymer (FIG. 24) further confirming the complete polymerization.

Peaks at 4.06 ppm and 5.8-5.9 ppm in NMR spectra were especially useful for estimation of the monomers ratio in the polymers (FIG. 5) found to be ca. 10.

In order to amend the length of the polymeric chains (Mw), the quantity of polymerization initiator (4,4′ -Azobis(4-cyanovaleric) acid) was varied. The polymers (samples 6 and 7) with increased quantity of initiator were synthesized next. Considering the best performance of the MSNs with coated with the polymer-1 in our US-controlled fluorescein release (vide infra), the ratio of the monomers in the reaction mixture was kept similar to the protocol used for its synthesis (FIG. 25).

The aggregation state of the polymers in ethanol and in aqueous solutions was examined by dynamic light scattering (DLS) at room temperature. It was found that all three polymers prepared in this work formed micelles with average size ca. 10-20 nm in ethanol, while in water only the large aggregates were detected (FIG. 6). Aggregation of the polymers at room temperature in water is consistent with the expected formation of large globules required for pores blocking.

The molecular weight (M_{w}) of the polymers were determined by Gel permeation chromatography (GPC). This analysis showed that the M_{w} values for copolymer-1 and copolymer-2 were 44000 and 33000 Da, respectively (FIG. 7 and FIG. 26).

FIG. 7. GPC RID analysis data for copolymer-1.

Since p(MEO2MA-co-THPMA) undergoes temperature-induced conformational changes, the temperature-dependent DLS signal relevant to its grafting on MSN samples has been studied. The aim of this experiment was to determine transition phase temperature. In our hands, at low temperature the polymer adopted coil conformation permitting a cargo of interest to enter mesopores of a particle. Increasing the temperature led to a likely conformational change of the polymeric chain. This transition resulted in a “collapsed” globular topology of the surface polymer to afford closed/partially closed pores on the surface of a MSN particle. As a next step, the phase transition temperature was determined (temperature that corresponds to the scattering intensity of 50% of the maximum @633 nm wavelength). The measurement was based on the effect of polymer's conformation change corresponding to the system reaching the phase transition temperature (lower critical solution temperature, LCST). At the LCST point the polymer conformation is expected to change to a hydrophobic state, where the molecules collapse and the compound becomes insoluble in water to result in the DLS signal. In our experimental setup, the temperature was increased by small increments in the range 10-45° C. and readings were taken after 2 min equilibration at each temperature point. For copolymer 1, the phase transition temperature was determined to be ca. 21-22° C. (FIG. 8). Based on this insight, the cargo loading in the subsequent experiments was performed at T<20° C., whereas for cargo release step the system was heated.

Immobilization of the polymer on the Rhodamine-labeled MSNs surface (polymer grafting) was performed via the carboxylic tether using N,N′-Dicyclohexylcarbodiimide and N-Hydroxysuccinimide The procedure of polymer grafting was repeated three times (several additions of the polymer to the reaction mixture for the same sample without intermediate sample isolation) in order to increase the quantity of the immobilized polymer. Powder XRD analysis suggested that the samples of polymer-coated MSNs do exhibit mesostructure ordering (FIG. 27). Furthermore, MSN samples treated with copolymers 1 or 2 showed characteristic adsorption bands in IR-spectra (ex., 1727 cm⁻¹ band that can be assigned to —O—CH₂—, FIG. 28).

For evaluation of cargo release the polymer-grafted MSNs were loaded with fluorescein in phosphate buffer solution (PBS) by 1 day stirring of the solution with MSNs at 4° C. The sample was filtered at 4° C. and washed with warm (50° C.) PBS in order to induce pores closing. The procedure of sample washing was repeated ca. 10 times to result in a colorless filtrate after sample washing. The resulting residue was put in polypropylene tube, charged with 10 mL of PBS buffer and treated by ultrasound. In the control, the same sample was placed in polypropylene tube and charged with 10 mL of PBS buffer without any ultrasound treatment. Both mixtures (ultrasound-treated and control) were heated at 37° C. for a predetermined time and a sample was taken for analysis (see FIGS. 9, 10, 12-14). MSNs were separated by centrifugation and fluorescence of the supernatant was measured using calibration curve (FIG. 29).

Both MSNs samples treated with copolymers 1 or 2 showed dramatically enhanced fluorescein release post ultrasound treatment compared to the controls. In our hands, the best difference was found for the MSNs treated with the polymer 1 (FIG. 9).

MSNs samples untreated with polymers were studied for comparison. Fluorescein release in all studied cases was 5-6 times higher than in the polymer-grafted samples, however no influence of US treatment on the cargo release was found (FIG. 10).

In order to find the mechanism for pores opening, the effect of ultrasound on polymer hydrolysis was studied. Specifically, the polymer solution of the polymer was subjected to sonication at 20 KHz and the products of hydrolysis were analyzed by ¹H NMR spectroscopy to reveal the formation of monomers and hydrolytic products (FIG. 11). Further, aqueous solution of the suspended polymer-1 and polymer-2 were treated by 20 kHz US for 10 minutes to discover a significant decrease in pH (‘acidification’) of the media. This change of pH was further enhanced by the increase in MW of the polymer used. For example, when 0.26 g of the polymer (both 1 and 2) in 30 mL of water were treated with ultrasound, the pH value of the solution decreased from 6.5 to 4.47. This effect was attributed to the formation of carboxylic groups due to hydrolysis of ester groups.

Scaled MSNs sample showed better difference (ca. 3-fold) in fluorescein release between the ultrasound treated samples and controls (FIG. 12).

The response of polymer-grafted MSNs sample to 1 MHz frequency ultrasound was further tested. The experiments were carried using protocol reported above. In contrast to the data obtained for the 20 KHz ultrasound protocol, the release of the model compound was enhanced modestly across multiple MSN samples (ca., 5% of increase vs baseline, FIG. 13). Based on these comparative data, the synthetic procedure was scaled up for synthesis of large batch of polymer-grafted MSNs (sample MSN-6). Both polymers 1 and 2 were immobilized on the MSN-6 sample and treated with the ultrasound at 20 KHz and 1 MHz, as described above. Fluorescein release @20 KHz ultrasound frequency was consistently higher (×2-3 times) compared to control experiment. On the other hand, treatment of the MSN-6 with 1MHz ultrasound was less efficient.

FIG. 13. Fluorescein release profiles for MSN-6 samples in PBS solution versus time with US exposure (1 MHz, 8 W or 20 kHz, ˜14 W) and without US.

In a separate experiment, the ultrasound treatment was performed at temperatures ranging between 2 and 55° C. Whereas the enhanced release of fluorescein under 1 MHz ultrasound treatment at higher temperatures was observed, it was somewhat lower compared to the effect of the 20 KHz frequency (FIG. 14).

FIG. 14. Fluorescein release profiles for MSN-6 samples in PBS solution versus time with US exposure (1 MHz, 8 W—at different t or 20 kHz, ˜14 W) and without US.

In order to increase the ultrasound intensity, a concentrating lens was manufactured (FIG. 30). The lens for US concentration is concave surface made of organic glass with a radius R of ca. 2.5 cm. It was expected to focus the ultrasound envelope because the speed of US waves in organic glass (polymethyl methacrylate) is higher than in the aqueous media and physiological matrices (2700 m/s and 1500 m/s, respectively). The photo of 1 MHz US, focused by similar lens, is shown on FIG. 15 (the photo was obtained by shadow method.

FIG. 15. Scheme of US focusing lens (a) and visualization of 1 MHz ultrasound focused by spherical lens with R=3 cm (b).

Certain embodiments of the present invention rely on ultrasound (US) for remote triggering and navigation of carriers implanted in living tissue. Other embodiments combine ultrasound with other external physical stimuli, non-limiting examples of which include: electromagnetic fields, phenomena, and effects; and thermodynamic phenomena and effects, including both temperature and pressure effects.

The terms “carrier device” and “carrier” herein denote any object that is implantable in biological tissue and is capable of carrying and releasing a medical payload (functional material) into the tissue. In some embodiments, the term “device” or the term “particle” are used to describe the carrier or the carrier device. The term functional material or“medical payload”, or equivalently the term “payload” used in a medical context is understood herein to include any substance or material of a medically-therapeutic or diagnostic nature. In certain embodiments, the medical payload or payload is equivalent to a “functional material” wherein the function is related to or directed toward treatment or for diagnostic purposes. The term “device” (with reference to a carrier) herein denotes a carrier which is constructed or fabricated by physical/chemical production/manufacturing techniques, including, but not limited to deposition, chemical reaction, chemical or physical binding, etching, lithography, thin-film technologies, deposition technologies, coating, molding, liquid and gas treatments, self-assembly, chemical synthesis and the like. The term “particle” in some embodiments of this invention is noted with reference to a carrier device. In other embodiment, the term “particle” is noted with reference to portions of the carrier device, e.g. to the porous particle.

In various embodiments of the present invention, carrier devices are miniaturized for implantation in biological tissues. The term “miniaturized” (with reference to a carrier) herein denotes a carrier of small size, including, but not limited to: carriers of millimeter to centimeter scale; carriers of micrometer (“micron”) scale, referred to as “carrier micro-devices”; carriers of nanometer scale referred to as “carrier nano-devices”. Not only are the carriers themselves of the size scales as indicated above, but the carriers' individual components are also of comparable scale. It is to be noted that certain carrier dimensions can be of different scales, e.g., a carrier may have one dimension in the nanometer range and another dimension in the micrometer range. All such miniatured devices are included in embodiments of this invention.

In one embodiment, porous materials of this invention comprise multiple pores. In one embodiment, porous materials of this inevntion are mesoporous materials and comprise pores ranging in size between 2 nm and 50 nm. In one embodiment, porous materials of this invention comprise mostly mesopores, and smaller amount of micro- and macro-pores. In one embodiment, porous materials of this inevntion comprise any combination of micropores, mesopores and macropores where micropores are in the range of up to 2 nm in diameter, mesopores diameters range between 2 nm and 50 nm and macropores are those larger than 50 nm in diameter. Porous particles of this inevntion are solid materials in one embodiment. Porous particles of this invention are stable in liquid as well as in a non-liquid environment in one embodiment. According to this aspect and in one embodiment, porous particles of this inventions are not vesicles or liposomes and differ from such particles in various properties, including being a multi-porous materials, being solid materials and being stable in liquid and in non-liquid environments. In one embodiment, porous particles of this invention comprise multiple pores. In one embodiment, the porous particle is a porous structure. In one embodiment, the porous particle has the shape of a sphere. In other embodiments, the porous particle/structure can assume any shape including rod, disc, box, pointed shape, oval, leaf, or any symmetric/assymmetric/partially symmetric shape.

In one embodiment, the propelling element/propelling component is attached to the porous particle. According to this aspect and in one embodiment, the propelling component is attached to the porous particle directly (not through the coating). In other embodiments, the propelling component is attached to the coating of the porous particle. In one embodiment, the porous particle is attached to the propelling component and only following this attachment, the porous particle is being coated by the coating material. In one embodiment, the porous particle is first coated and then attached to the propelling component. In one embodiment, the propelling component is attached to the external surface of the porous particle. In one embodiment, the propelling component is attached to an internal region of the porous particle. In one embodiment, the propelling component is a material that is bound to the pores of the porous particle.

In one embodiment, the propelling component is a magnetic component. According to this aspect and in one embodiment, the magnetic component comprise a magnetic material. In one embodiment, the magnetic component is responsive to magnetic and/or electric fields. The magnetic competent allows navigation of the device in one embodiment. In one embodiment, when magnetic or electromagnetic field is applied, the magnetic component respond to the field. Accordingly, the magnetic component can be moved from one location to the other by applying external field. The magnetic component is used to direct the device to a certain location for controlled release of the functional material from the device. Application of external field (external stimulus) is used to drive the device to a desired location.

In some embodiments, the coating material is US sensitive. In one embodiment, upon exposure to US, the coating material changes its shape/conformation. In some embodiments, the coating material undergoes chemical modifications as exemplified by but not limited to hydrolysis in response to US.

In some embodiments, the US-sensitive material undergoes a structural modification in response to US. According to this aspect and in one embodiment, prior to the application of US, the coating material is in a shape or form that is closed, i.e. functional material cannot penetrate or transfer through the coating material. Accordingly, when the coating material of this structure coats the porous particle, functional material that resides in pores of the porous particle is confined in the particle and cannot be released. However, when the coating material undergoes a structural modification (in response to US for example), it becomes perforated or changes its shape or peels, such that functional material can be transferred from the porous particle to the surrounding tissue.

A Globule Structure

In one embodiment, the coating material is a dry film of a non-polymeric material. In one embodiment, the device of this invention can be placed or directed to a certain tissue and the functional material is released in that tissue. In other embodiments, the device of this invention can be placed or directed to a certain tissue and the functional material is released to an adjacent (other) tissue.

In one embodiment, one device is being used for release of a functional material in a tissue, a blood stream, lymphatic, biliary or cerebrospinal fluid flow. In other embodiments, two or more devices are used for the release of functional material in a tissue. According to this aspect and in one embodiment, the two or more devices are operated to release the same functional material. In other embodiments, different devices in a fleet of devices contain different functional materials that are being released as needed. The release of one or more functional material from two or more devices is conducted in parallel. For example, at the same time some devices release a first material and other devices release a second material. The use of multiple devices with one or more functional material results in enhanced therapeutic or diagnostic effect in one embodiment.

In one embodiment, the porous material is silica or alumina. In one embodiment, the porous material (porous particle) comprise silica or alumina In one embodiment, the porous particle comprises SiO₂, Al₂O₃, or other inorganic porous materials. In some embodiments, the porous material is an organic porous material or an organic/inorganic porous material.

In one embodiment, the average size of the porous particle ranges between 10 nm and 300 nm or between 10 nm and 1000 nm or between 50 nm and 250 nm.

In one embodiment, the total volume of the pores of the porous particle ranges between 0.5 cm³/g and 5.0 cm³/g. In some embodiments, any other value of total pore volume associated with a certain porous material is applicable in embodiments of this invention.

In one embodiment, the pore diameter ranges between 3 nm and 10 nm. In one embodiment, the pore diameter ranges between 5 nm and 50 nm. In one embodiment, the pore diameter ranges between 2 nm and 50 nm. In one embodiment, the pore diameter ranges between 1 nm and 50 nm, between 1 nm and 75 nm, between 1 nm and 100 nm. In some embodiment, The ranges above are given for all of the pores. In other embodiments, the ranges above are given for the majority of the pores in a certain area/particle/material. Small amount of smaller or larger pores may exist in porous materials of the invention. For example, less than 20%, less than 10%, less than 5%, less than 1%, less than 0.1%, or less than 0.01% of the pores may be outside the ranges given above for the size/diameter of the majority of the pores.

In one embodiment, this invention provides a carrier device for implanting in a biological tissue for precise delivery and release of a functional material in the tissue or in another tissue, the carrier device comprising:
a porous particle coated by US-sensitive coating material;
a functional material; and
a propelling component;
   wherein, the functional material resides in the pores of the porous material; and wherein the propelling component is attached to the porous particle. In one embodiment, the frequency of the US ranges between 10 and 100 KHz. This frequency range is the range to which the coating material is sensitive to in some embodiments. Additional representative examples of the ultrasound sensitive polymers that could be used include high density polyethylene, low density polyethylene, linear low density polyethylene, polypropylene, polyamide 6, PS and PMMA. According to this embodiment, when US at that frequency range is applied, the US-sensitive coating material changes its shape/conformation as described herein above.

In one embodiment, the particle is a microparticle, a nanoparticle or a combination thereof.

In one embodiment, the particle is a nanoparticle, where the largest dimension of the particle is in the nanometer range. In some embodiment, the particle is a microparticle where the largest dimension of the particle is of a micrometer range. In some embodiments, the particle size ranges between 500 nm and 1000 nm and it can be considered both as nanoparticle and as microparticle. In some embodiments, a certain dimension (e.g. length) is in the micrometer range, while another dimension (e.g. thickness) is in the nanometer range. Such particle may be considered as micro.nano particle. All such combinations are included in embodiments of this invention.

In one embodiment as described herein, the carrier device of this invention comprises a porous particle coated by US-sensitive coating material. In some embodiments, the particle is fully-coated by the coating material. In other embodiments, areas from which functional material cannot be released upon US application are not covered/coated by the coating material. For example, in some embodiments, the propelling component is attached to a region of the surface of the particle. According to one embodiment, this region is not coated by the coating material. Accordingly, in some embodiments, the coating material coats a portion of the porous particle. In some embodiments, the coating material coats at least 50%, at least 60%, at least 70%, at least 805, at least 90%, at least 95%, at least 98%, at least 99%, at least 99.5%, at least 99.9% or at least 99.99% of the external surface of the porous particle.

In some embodiment, this invention provides a method of treating a subject, the method comprises:
inserting the device of claim 1 into the subject;
applying external stimuli to the device.

In some embodiments, the particle is first inserted into the subject, it is then transferred to a desired location in a tissue and following transfer, the functional material is released from the particle. The transfer of the particle to the desired location, the release of the functional material or a combination thereof is controlled by an external stimulus. In some embodiments, the stimulus that controls particle transfer is different from the stimulus for functional material such that during transfer, functional material is secured and following transfer functional material is released. In some embodiments, stimulus for transfer and stimulus for release can be applied at the same time for dynamic release of functional material along a certain path. In some embodiment, the stimulus for transfer and for release are different in nature (e.g. one is US and the other magnetic-based). In other embodiments, the stimulus for transfer and the stimulus for release are different in magnitude/value (for example transfer is induced by US of one frequency while release is induced by US of another frequency).

Cargo refers to functional material or payload in some embodiments, carrier device is viewed as a container in some embodiments.

### EXAMPLES

### Example 1

### 1. Synthesis of Starting Compounds and Materials

### 1.1. Mesoporous Silica Nanoparticles (MSNs)

1 g of cetyltrimethylammonium bromide (CTAB), 480 mL of H₂O (Millie-Q) and 3.5 mL of NaOH (2 M) were added to a 1000 mL round-bottom flask. The mixture was heated to 80° C. and stirred at 600 rpm. Then, 5 mL of TEOS were added dropwise at 0.25 mL/min rate with a pump. The white suspension obtained was magnetically stirred for further 2h at 80° C. Then, the reaction mixture was centrifuged and washed with water and ethanol. Finally, the template was removed by ionic exchange using a solution of ammonium nitrate (10 mg/mL) in ethanol (95%) at 70° C. overnight under magnetic stirring. The nanoparticles were collected by centrifugation, washed with ethanol three times and dried under vacuum overnight.

### 1.2. Rhodamine B-Labelled Mesoporous Silica Nanoparticles (MSNs).

1 g of CTAB, 480 mL of H₂O (Millie-Q) and 3.5 mL of NaOH (2 M) were added to a 1000 mL round-bottom flask. The mixture was heated to 80° C. and stirred at 600 rpm. 1 mg of Rhodamine-B isothiocyanate with 2.24, of (3-aminopropyl)triethoxysilane (APTES) in 100 μL ethanol for 2h was mixed and resulted solution added into 5 mL of TEOS, then mixture were added dropwise at 0.25 mL/min rate with a pump. The white suspension obtained was magnetically stirred for further 2 h at 80° C. Then, the reaction mixture was centrifuged and washed with water and ethanol. Finally, the template was removed by ionic exchange using a solution of ammonium nitrate (10 mg/mL) in ethanol (95%) at 70° C. overnight under magnetic stirring. The nanoparticles were collected by centrifugation, washed with ethanol three times and dried under vacuum overnight.

### 1.3. US-Responsive Monomer, Tetrahydropyranyl Methacrylate (THPMA)

Methacrylic acid (8 g), pyridine (0.3 mL), and p-toluenesulfonic acid (0.7 g) were dissolved in 80 mL of dichloromethane (FIG. S7a). Dihydropyran (0.162 mol) was slowly added at room temperature. After stirring overnight, the solution was filtered by a short silica column. The solution was extracted by water and brine three times. Finally, the solvent was removed under vacuum to yield THPMA.

1.4. US-Responsive Copolymer p(MEO₂MA-co-THPMA).

The copolymer, poly(2-(2-methoxyethoxy)ethylmethacrylate-co-2-tetrahydropyranyl methacrylate), p(MEO2MA-co-THPMA), was synthesized by free radical polymerization from MEO2MA (temperature-responsive monomer) and THPMA (ultrasound-responsive monomer). MEO2MA and THPMA at different molar ratios (0.01 mol in total) were placed in a seal vial and purged with nitrogen. 16 mL of DMF were added under inert atmosphere and the solution was placed at 80° C. under magnetic stirring. 1 mL of DMF with 0.003 mmol of initiator 4,4′-Azobis(4-cyanovaleric acid (ABCVA) was added. The reaction was carried out overnight. Then, the polymer was precipitated in cold diethylether, separated by centrifugation and washed 3 times with diethylether followed by evaporation of the solvent.

### 1.5. Polymer Grafted MSN Nanoparticles.

0.3 g of carboxylic acid-terminated poly(MEO2MA-co-THPMA), 11 mg of N,N′-Dicyclohexylcarbodiimide (DCC) and 6 mg of N-Hydroxysuccinimide (NHS) were added to a glass vial. The vial was purged with nitrogen and 2 mL DMF were added. Then, under N₂ atmosphere and with magnetic stirring, DMF (1 mL) with 8 μl APTES were added. The solution was stirred overnight (Solution 1, sililated copolymer solution). Then, 1 mL of Solution 1 was added dropwise to 20 mL of toluene containing 50 mg of MSNs under vigorous stirring. The reaction medium was heated under reflux. After 4 h, another mL of Solution 1 was added. 4 h later, the remaining Solution 1 was added. The reaction was left under vigorous stirring for 24 h. Then, the hybrid MSNs were collected by centrifugation and washed with toluene, DMF (twice), cold water (twice) and ethanol Afterwards, the nanoparticles were dried under vacuum for 16 h. Before dye loading the samples were thoroughly washed by toluene, DMF (twice), water and ethanol and dried in vacuum at 60° C.

### 1.6. Determination of Phase Transition Temperatures (LCST) of Polymer Grafted MSN Nanoparticles by Dynamic Light Scattering.

LCST was determined by Dynamic Light Scattering (DLS) by means of the drastic change in the scattering intensity obtained by precipitation of the polymer at the LCST (determined as the temperature at which the scattering intensity is 50% of the maximum) Measurement of the LCST was performed using a Zetasizer Nano-S (Malvern Instruments) equipped with a 633 nm “red” laser. To determine the transition temperature, the temperature dependence of the scattering intensity at 90° from 1 mL of solution in a glass cuvette was measured. The temperature was increased by discrete temperature increments in the range 10-45° C., and the readings were taken after 2 mL equilibration at each temperature.

### 1.7. Cargo Loading and Release for Ultrasound-Responsiveness of the Copolymer-Grafted MSN

Cargo loading: 20 mg of nanoparticles were placed in a glass vial with a septum and dried at 80° C. under vacuum for 24 h. Then, the vial was placed at 4° C. with magnetic stirring and 5 mL of cargo solution (20 mg/mL, fluorescein in PBS) were added and the suspension was stirred at 4° C. for 24 h. After that time, the sample was filtered and washed two times with previously hot PBS (50° C.) in order to remove the cargo absorbed on the external surface. Finally, the products were dried under vacuum at 25° C.

Cargo Release: The polymer-grafted MSNs were loaded by fluorescein in phosphate buffer solution (PBS) by 1 day stirring of the solution with MSNs at 4° C. The sample was filtered at 4° C. and washed by warm (50° C.) PBS in order to induce pores closing due to thermoresponsivity of the copolymer. The procedure of sample washing was repeated 8-11 times, until getting of colorless solution after sample washing for total release of FL from external surface (only the fluorescein trapped in pores had to remain in the sample). Then the sample was put in polypropylene tube, charged with 10 mL of PBS buffer and treated by US. Notably, US treatment causes solution heating, however in our belief this was not significant in view of further solution heating on the next step. In control experiment the sample was placed in polypropylene tube and charged with 10 mL of PBS buffer without any US treatment. Then both solution (treated by the US and control) were heated at 37° C. for certain time, and after specified heating period sample was taken for analysis. MSNs were separated by centrifugation, and fluorescence of the clear solution was measured using calibration curve, Fluorescein: λ_{exc} 490, λ_{cm} 514 nm (FIG. 29).

### 1.8. Polymer Hydrolysis.

Specific quantity of the polymer (from 0.07 to 0.26 g) were suspended in 30 mL of water. The suspension was treated by 20 kHz US during 10 minutes, then pH of the solution was measured by pH-meter.

### Example 2

### 2. Methods

Powder diffraction was measured on D8 ADVANCE, Bruker AXS diffractometer. Nitrogen adsorption for determination of samples surface and volume was measured using Sorptomatic 1990 (Thermo Electron) instrument at 77 K. Dynamic light scattering was measured using Zetasizer Nano-S, Malvern device. Transmission electronic images were obtained with TEM (PEM-125K, Selmi) microscope, elements analysis (CHN) was performed on CarloErba 1106 analyzer. Thermal analysis was carried out on Q-1000, MOM derivatograph. IR spectra were measured on Spectrum One Perkin Elmer FTIR spectrometer in KBr disks. UV-Vis spectra were measured on Specord 210, Analytik Jena spectrometer, and luminescent spectra of solid samples and solutions were measured on LS55, Perkin Elmer luminescent spectrometer. ¹H NMR and ¹³C spectra were measured on Unity Plus 400, Varian NMR spectrometer.

FIG. 16. TEM images of MSN-1 sample (mesopore ordering with d-4.5 nm, Dₚₒᵣₑ˜2.8 nm is shown)

FIG. 17 XRD patterns of MSNs samples.

FIG. 18 Thermal analysis of MSNs samples.

FIG. 19 (a) (b) Intra-particle (19a) and inter-particle (19b) pore size distribution for MSNs-1 (1), MSNs-2 (2), MSNs-3 (3), MSNs-4 (4) and MSNs-5 (5) samples calculated from N₂ adsorption isotherms.

TABLE 1
Sorption characteristics of MSN samples.

| | V_{mic}¹, | Vₘₑ¹, | Sₘₑ¹, | Dₘₑ¹, | \|Δμ₀\|, | Sₑₓₜ¹, | S_{BET}, | Vₜ, |
|---|---|---|---|---|---|---|---|---|
| Sample | cm³/g | cm³/g | m²/g | nm | kJ/mol | m²/g | m²/g | cm³/g |
| MSN-1 | 0 | 0.658 | 773 | 2.85 | 8.9 ± 0.18 | 44 | 817 | 1.007 |
| MSN-2 | 0 | 0.839 | 952 | 2.96 | 9.1 ± 0.19 | 56 | 1007 | 1.320 |
| MSN-3 | 0 | 0.636 | 973 | 2.19 | 8.6 ± 0.32 | 67 | 1044 | 0.896 |
| MSN-4 | 0 | 0.867 | 951 | 3.02 | 9.8 ± 0.26 | 80 | 1033 | 1.360 |
| MSN-5 | 0 | 0.800 | 915 | 2.91 | 9.5 ± 0.24 | 72 | 989 | 1.094 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A carrier device for implanting in a biological tissue or physiological flow as exemplified but not limited to blood, lymph, bile, cerebrospinal fluid for precise delivery and release of a functional material in said tissue or in another tissue, the carrier device comprising:
a porous particle coated by US-sensitive coating material;
a functional material; and
a propelling component;
wherein, said functional material resides in the pores of said porous material; and
wherein said propelling component is attached to said porous particle.

2. The device of claim 1, wherein said propelling component is a magnetic component.

3. The device of claim 1, wherein said coating material is a polymer.

4. The device of claim 1, wherein said propelling component and said coating materials are responsive to external stimuli.

5. The device of claim 4, wherein:
said coating material is sensitive to ultrasound (US) stimuli; and
said propelling component is responsive to stimuli selected from US, magnetic, electric, electromagnetic, thermal, electromagnetic radiation or a combination thereof.

6. The device of claim 5, wherein application of said stimuli to said propelling component propels said device.

7. The device of claim 1, wherein said US-sensitive material undergoes a structural or chemical modification in response to US.

8. The device of claim 7, wherein said structural modification comprises a change from coil conformation to globular conformation structure.

9. The device of claim 1, wherein said US-sensitive coating material changes its chemical structure, molecular weight, length, shape or topology or detaches from said particle or ruptures or becomes perforated in response to said external US stimuli.

10. The device of claim 1, wherein said porous material is silica or alumina

11. The device of claim 1, wherein the average size of said porous particle ranges between 10 nm and 1000 nm.

12. The device of claim 9, wherein the average size of said porous particle ranges between 100-150 nm or between 100-200 nm or between 50-100 nm or between 10-50 nm or between 2-50 nm or between 60-130 nm or between 70-150 nm or between 30-60 nm.

13. The device of claim 1, wherein the BET specific surface area of the porous particle ranges between SBET 817-1044 m²/g.

14. The device of claim 1, wherein the total volume of the pores of the porous particle ranges between 0.5 cm³/g and 5.0 cm³/g.

15. The device of claim 1, wherein the pore diameter ranges between 3 nm and 10 nm.

16. The device of claim 1, wherein the frequency of said US ranges between 10 and 100 KHz.

17. The device of claim 1, wherein the frequency of said US is 20KHz.

18. The device of claim 1, wherein said US-sensitive material does not undergo structural modification in response to US in a MHz frequency range.

19. The device of claim 1, wherein said functional material is an organic compound, a polymer, a composite or a combination thereof.

20. The device of claim 1, wherein said functional material comprises small molecules, biological materials, gene therapy components, antisense oligonucleotides, aptamers, peptides, peptoids, endogenous or engineered cells, oncolytic viruses or radiation therapy materials.

21. The device of claim 20, wherein said gene therapy components comprise CRISPR/Cas9 or viral vector-based agents.

22. The device of claim 1, wherein said particle is a microparticle, a nanoparticle or a combination thereof.

23. The device of claim 3, wherein said polymer is a copolymer, comprising or consisting of: 2-(2-methoxyethoxy)ethylmethacrylate and tetrahydropyranyl methacrylate-poly(2-(2-methoxyethoxy)ethylmethacrylate-co-2-tetrahydropyranyl methacrylate), p(MEO2MA-co-THPMA), high density polyethylene, low density polyethylene, linear low density polyethylene, polypropylene, polyamide 6, PS and PMMA.

24. The device of claim 23, wherein said polymer comprises a carboxy end group.

25. The device of claim 24, wherein said porous particle comprise (3-aminopropyl) triethoxysilane (APTES) and wherein said carboxy group of said polymer binds to the NH₂ group of said APTES for covalent immobilization of said polymer on said porous particle.

26. The device of claim 3, wherein said polymer is temperature sensitive.

27. The device of claim 26, wherein said polymer changes conformation in response to a temperature change.

28. The device of claim 27, wherein said conformational change comprises coil structure at temperatures below 20-30° C. and globular structure at temperatures above 20-30° C.

29. A system comprising:
the device of claim 1; and
a remote unit;
wherein said remote unit is configured to apply external stimuli to said device.

30. The system of claim 29, wherein said external stimuli comprises US.

31. The system of claim 29, wherein:
said coating material changes its chemical structure, molecular weight, length, shape or topology, or detaches from said particle ruptures or becomes perforated in response to said external stimuli; or
said propelling component is driven in response to said external stimuli; or
a combination thereof.

32. A method for operating a device, said method comprising:
providing a carrier device comprising:
a porous particle coated by US sensitive coating material;
a functional material; and
a propelling component;
wherein, said functional material resides in the pores of said porous material; and
wherein said propelling component is attached to said porous particle;
applying external stimuli to said device.

33. The method of claim 32, wherein said coating is responsive to said external stimuli.

34. The method of claim 32, wherein said stimuli is US, magnetic or a combination thereof.

35. The method of claim 32, wherein:
said coating polymer changes its chemical structure, molecular weight, length, shape or topology, or detaches from said particle ruptures or becomes perforated in response to said external stimuli, such that said functional material is released from said particle in response to said external stimuli; or
said propelling component is driven in response to said external stimuli; or
a combination thereof.

36. The method of claim 35, wherein:
said coating material is responsive to US and said propelling component is responsive to magnetic stimuli; or
said coating material is responsive to US of a first frequency and said propelling component is responsive to US of a second frequency.

37. The method of claim 32, wherein said functional material is an organic compound, a polymer, a copolymer, a composite or a combination thereof.

38. The method of claim 32, wherein said coating material comprising a polymer or copolymer.

39. The method of claim 32, wherein said porous particle is a microstructure, a nanostructure or a combination thereof.

40. The method of claim 32, wherein said propelling component comprises a magnetic component.

41. A method of producing the device of claim 1, said method comprising:
providing or constructing a porous particle;
filling the pores of said porous particle with a functional material;
coating said porous particle with a US-sensitive coating material.
binding a propelling component to said porous particle, to said coating material or to a combination thereof.

42. A method of treating a subject, said method comprises:
inserting the device of claim 1 into said subject;
applying external stimuli to said device.

43. The method of claim 42, wherein said inserting the device comprises inserting the device into a certain tissue or physiological compartment within said subject.

44. The method of claim 42, wherein said external stimuli comprises:
magnetic/electric or electromagnetic stimuli to propel the device to a defined location within the subject; or
US stimuli to induce release of said functional material from said porous particle; or
a combination thereof.

45. The method of claim 42, wherein following application of said external stimuli, said functional material interacts with said tissue or with component(s) of/in said tissue.

46. The method of claim 45, wherein said interaction results in a therapeutic effect, a diagnostic effect or a combination thereof.

47. The method of claim 42, further comprising imaging the location of said device within said subject.

48. The method of claim 42, wherein said propelling component is a magnetic component.
